(19) United States
(12) Patent Application Publication
(10) Pub. No.: US 2005/0026912 A1
(43) Pub. Date: Feb. 3, 2005

(54) Method of treating restless leg syndrome

(76) Inventor: Joel C. Morgenlander, Durham, NC (US)

Correspondence Address: MYERS BIGEL SIBLEY & SAJOVEC, PO BOX 37428, RALEIGH, NC, 27627, US

(21) Appl. No.: 10/932,415

(22) Filed: Sep. 2, 2004

Related U.S. Application Data
(62) Division of application No. 10/341,643, filed on Jan. 14, 2003, now abandoned.
(60) Provisional application No. 60/363,583, filed on Mar. 12, 2002.

(51) Int. Cl. A61K 31/5513; A61H 1/00; A61H 1/02

(52) U.S. Cl. 514/221; 601/1; 514/567

(57) ABSTRACT

A method of treating restless leg syndrome (RLS) in a patient in need of such treatment includes applying positive pressure to an extremity of the patient at a magnitude and for a duration sufficient to combat RLS. In certain embodiments, the method comprises applying a pressure at a magnitude of between about 5 and 80 mm Hg to the extremity of the patient. In other embodiments, the method comprises applying the pressure intermittently for intervals of between about 5 seconds and 30 minutes, and may comprise applying such pressure between periods of rest of between about 10 seconds and 5 minutes.

### RELATED APPLICATIONS

This application claims priority from U.S. Provisional Application No. 60/363,583 filed Mar. 12, 2002, the disclosure of which is incorporated herein in its entirety by reference.

### FIELD OF THE INVENTION

The invention relates generally to the treatment of restless leg syndrome.

### BACKGROUND OF THE INVENTION

Restless Leg Syndrome (RLS) is a medical condition that causes the patient to have unpleasant limb sensations, particularly in the legs. Discomfort is particularly prevalent when the patient is at rest and often forces the patient to stand or walk frequently to relieve the discomfort. The symptoms are especially troublesome as the patient is trying to sleep (which can result in sleep onset or sleep maintenance insomnia), but may also occur when the patient is sitting, particularly for long periods, such as in an automobile, airplane, theater, classroom or church.

The restlessness is typically characterized by sensations of deep-seated tingling, burning, aching, or the like. These sensations contribute to the patient's compulsion to move the affected limbs, and forcing the limbs to remain still may cause an increase in the discomfort and/or involuntary jerking of the limbs. In some instances (about 80 percent) of RLS cases, the patient will experience periodic limb movements of sleep (PLMS), which are repeated flexion movements of the legs that last from 0.5 to 5 seconds and occur semirhythmically at intervals of 20 to 24 seconds. PLMS can cause arousals that fragment sleep and result in daytime sleepiness.

Typically, RLS is treated with minor changes in lifestyle, such as minimizing caffeine, alcohol and nicotine before bed, or through pharmacologic treatments, such as benzodiazepines, dopamine agonists, carbidopa-levodopa, anti-convulsants, and opioids. Shortcomings with these treatments include the typical side effects of medications, such as drowsiness during waking hours or cognitive impairment, reduction in the efficacy the medications over time, and worsening of symptoms at certain periods of the day.

### SUMMARY OF THE INVENTION

As a first aspect, the present invention is directed to a method of treating RLS in a patient in need of such treatment. The method comprises applying positive pressure to an extremity of the patient at a magnitude and for a duration sufficient to combat restless leg syndrome.

In certain embodiments, the method comprises applying a pressure at a magnitude of between about 5 and 80 mm Hg to the extremity of the patient. In other embodiments, the method comprises applying the pressure intermittently for intervals of between about 5 seconds and 30 minutes, and may comprise applying such pressure between periods of rest of between about 10 seconds and 5 minutes.

As a second aspect, the present invention is directed to an apparatus for treating RLS. The device comprises: a sleeve configured to fit over an extremity of a patient with RLS and a pressure source connected to and in fluid communication with the sleeve that is configured to apply pressure to the extremity at a magnitude and for a duration sufficient to combat RLS. In some embodiments, such a device may apply intermittent pressure, and may include a controller that monitors the pressure application schedule, duration and magnitude.

### BRIEF DESCRIPTION OF THE FIGURES

FIG. 1 is a schematic perspective view of an embodiment of a device of the present invention attached to the leg of a patient.

FIG. 2 is a schematic perspective view of another embodiment of a device of the present invention.

### DETAILED DESCRIPTION OF THE INVENTION

The present invention will now be described more fully hereinafter, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

As noted above, the present invention is directed to the treatment of RLS. As used herein, the terms “restless leg syndrome” and “RLS” are intended to refer to a medical condition characterized by unpleasant limb sensations that are typically precipitated by rest and relieved, at least partially, by activity. The clinical features, etiologic factors, pathogenesis and diagnosis of RLS are described in M. Sibler, Concise Review for Primary Care Physicians: Restless Leg Syndrome, 72 Mayo Clin. Proc. 261-264 (1997).

The treatment is typically practiced on human subjects, but may be practiced on veterinary subjects, including other primates and other mammals, that suffer from RLS. For human subjects, pressure is typically applied to one or both of the legs of the patient, but may be applied to any extremity (e.g., the subject's arms) affected by RLS symptoms. Also, pressure may be applied to one or more portions of the extremity. For example, in some embodiments pressure may be applied only to the calf of the patient, while in other embodiments pressure may be applied to both the calf and the thigh of the patient.

Pressure may be applied continuously or intermittently for a duration sufficient to combat RLS. If pressure is applied intermittently, it may be applied for durations of between about 5 seconds and 30 minutes, and in some embodiments for between about 10 and 60 seconds. These periods of application typically fall between periods of rest (i.e., non-application) having durations of between about 10 seconds and 5 minutes. The periods of application may be uniform or non-uniform, as may be periods of non-application. Alternatively, the pressure may be applied for intermittent periods of varying pressure (e.g., alternating periods of elevated pressure and reduced pressure) rather than periods of rest occurring between application periods.

Pressure should be applied at a magnitude sufficient to combat RLS, which is typically between about 5 and 80 mm Hg, and in some embodiments between about 25 and 60 mm Hg. The pressure magnitude may be constant during each application period or may increase and/or decrease during that period. Also, pressure magnitude may vary between different pressure application periods. Further, pressure magnitude may differ for different sections of the extremity; for example, higher pressure may be applied at the ankle and lower pressure applied to the calf. Preferably, the magnitude of the pressure does not exceed a level that would cause the arteries in the patient's circulatory system to collapse.

The application of pressure may be carried out at any time that doing so combats RLS. In some embodiments of the invention, the application of pressure may occur as the patient is sleeping and/or is attempting to fall asleep.

It may be desirable to combine the application of pressure with other treatments for RLS. For example, it may be desirable to treat the patient with the application of pressure as described above as well as with the administration of a medicament believed to combat RLS. Exemplary medicaments include those selected from the group consisting of: benzodiazepines, dopamine agonists, carbidopa-levodopa, anti-convulsants, and opioids. Other exemplary medicaments are described in U.S. Pat. No. 6,001,861 to Oertel et al.; U.S. Pat. No. 5,945,424 to Schmidt; U.S. Pat. No. 6,114,326 to Schueler; and U.S. Pat. No. 5,759,198 to Karell.

In practicing the methods of the invention, a variety of factors known to those skilled in the art may be considered. Such factors include the age, size, gender, and general condition of the patient, and the like.

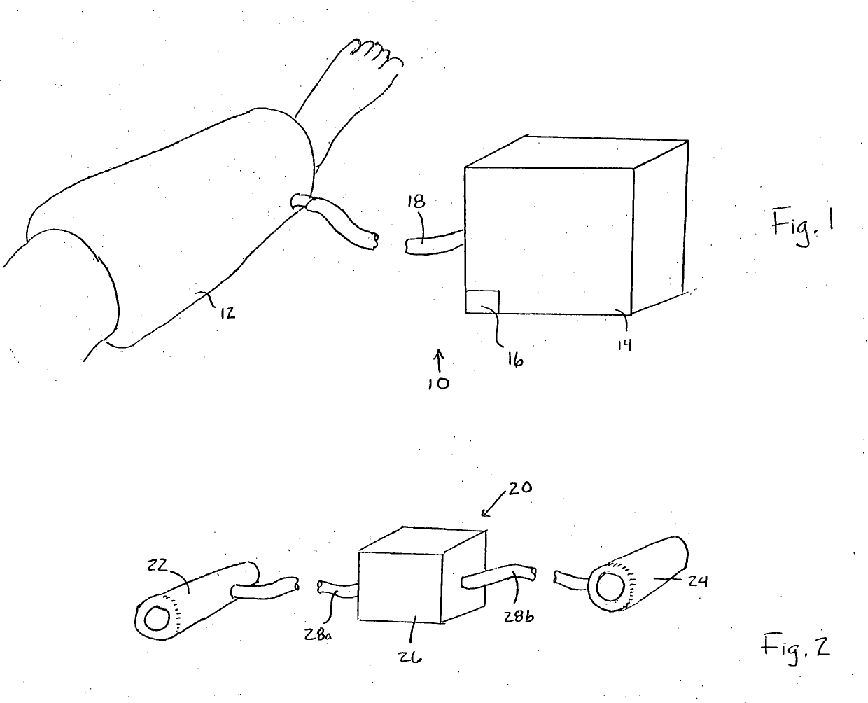

Treatments may be carried out with a device such as that illustrated in FIG. 1. The device shown therein, designated broadly at 10, includes a pressure sleeve 12 and a pressure source 14. The pressure sleeve 12, which is generally cylindrical, is configured to receive an appendage of a patient within its lumen and applies pressure thereto. The sleeve may extend the full length of the patient's extremity, or may extend over only a portion of its length (for example, only from the ankle to the knee). The sleeve 12 may be contiguous about its cross-section, such that it may be slipped over the extremity, or it may open to a flat form that can be wrapped around the extremity and attached to itself (for example, with hook-and-loop straps, zippers or buckles).

In the illustrated embodiment, the sleeve 12 is inflatable, and pressure is applied to the appendage through introduction of a fluid (such as air) to the sleeve 12. Such a sleeve may have a single cylindrical chamber or multiple chambers for receiving fluid. The sleeve 12 is typically formed of a material that is sufficiently flexible to conform to the patient's appendage; if the sleeve 12 is to apply pressure through fluid introduction, it should also be sufficiently watertight or airtight to allow the pressurizing fluid to be retained within the sleeve 12. Exemplary materials for sleeves include polymeric materials, such as vinyl, polyethylene, polypropylene, and the like.

The pressure source 14 is connected to and in fluid communication with the sleeve 12 through tubing 18. The pressure source 14 is configured to apply sufficient pressure to the patient's extremity to combat RLS (see the description above for appropriate magnitude, duration, and schedule of pressure application). Pressure may be created in the pressure source 14 through a rotary pump, one or more piston/cylinder combinations, or other positive pressure-inducing configurations. The pressure source 14 may be configured to provide a constant pressure, or may be adjustable in magnitude, duration, and schedule. A controller 16 may optionally be operatively associated with the pressure source 14 to control one or more of these pressure variables.

Those skilled in this art will appreciate that other techniques for the application of pressure to the patient's extremities may also be suitable for use with the present invention. For example, pressure may be applied through controlled mechanical constriction or shrinkage of the sleeve.

It should also be recognized that devices of the present invention may be configured to include more than one sleeve. FIG. 2 illustrates a device 20 having two sleeves 22, 24 attached to a pressure source 26 through tubing 28a, 28b. A controller 30 individually controls the characteristics of the pressure applied to the sleeves 22, 24. A device of this configuration can enable the patient to treat multiple extremities at once; for example, both legs can be treated as the patient sleeps.

The invention will now be described in greater detail in the following non-limiting example.

### EXAMPLE 1

A female patient, approximately age 60, was undergoing therapy for stroke. A venous compression system (provided by Venodyne®, Columbus, Miss.) was employed as prophylaxis for deep vein thrombosis (DVT), with a calf-length sleeve being attached to each of the patient's legs. Pressure of 45 mm Hg was applied for 12 second periods (45 seconds rest) while the patient slept. The patient, who had a history of RLS, reported marked improvement in the condition after two nights of application.

### EXAMPLE 2

A male patient, approximately age 50, was admitted for a brain biopsy. As prophylaxis for deep vein thrombosis (DVT), the venous compression system described in Example 1 was employed, with a calf-length sleeve being attached to each of the patient's legs. Pressure of 45 mm Hg was applied for 12 second minute periods (45 seconds rest) as the patient slept. The patient, who had a history of RLS, reported marked improvement in the condition after one night's sleep.

The foregoing is illustrative of the present invention and is not to be construed as limiting thereof. Although exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

1. A method of treating restless leg syndrome in a patient in need of such treatment, the method comprising applying positive pressure to an extremity of the patient at a magnitude and for a duration sufficient to combat restless leg syndrome.

2. The method defined in claim 1, wherein the positive pressure is applied intermittently.

3. The method defined in claim 2, wherein the intermittent positive pressure is repeatedly applied for a duration of between about 10 and 60 seconds.

4. The method defined in claim 2, wherein the intermittent positive pressure is applied between periods of non-application of between about 10 seconds and 5 minutes.

5. The method defined in claim 1, wherein the positive pressure is applied at a magnitude of between about 5 and 80 mm Hg.

6. The method defined in claim 1, wherein the positive pressure is applied to at least one leg of the patient.

7. The method defined in claim 5, wherein the positive pressure is applied to both legs of the patient.

8. The method defined in claim 1, further comprising administering a medicament to the patient, the medicament being selected from the group consisting of: benzodiazepines, dopamine agonists, carbidopa-levodopa, anti-convulsants, and opioids.

9. The method defined in claim 1, wherein the application of pressure is carried out on a sleeping subject.

10. A method of treating restless leg syndrome in a patient in need of such treatment, the method comprising applying intermittent positive pressure to at least one leg of the patient, the pressure being applied at a magnitude of between about 5 and 80 mm Hg, and the pressure being repeatedly applied for a duration of between about 5 seconds and 30 minutes.

11. The method defined in claim 10, wherein the intermittent pressure is applied to both legs of the patient.

12. The method defined in claim 10, further comprising administering a medicament to the patient, the medicament being selected from the group consisting of: benzodiazepines, dopamine agonists, carbidopa-levodopa, anti-convulsants, and opioids.

13. The method defined in claim 10, wherein the application of pressure is carried out on a sleeping subject.

14-19 Canceled
